(19) United States
(12) Patent Application Publication
(10) Pub. No.: US 2025/0360464 A1
(43) Pub. Date: Nov. 27, 2025

(54) METHOD OF FILTERING SOLIDS FROM A SOLUTION DERIVED FROM PLASMA

(71) Applicant: CSL BEHRING (AUSTRALIA) PTY LTD, Melbourne (AU)

(72) Inventors: Karl MCCANN, Melbourne (AU); Zhenghui XU, Melbourne (AU); Per HANSEN, Melbourne (AU); Jiaxi ZHANG, Melbourne (AU)

(73) Assignee: CSL BEHRING (AUSTRALIA) PTY LTD, Melbourne (AU)

(21) Appl. No.: 18/869,637

(22) PCT Filed: May 31, 2023
(86) PCT No.: PCT/AU2023/050464

§ 371 Date: Nov. 26, 2024

(30) Foreign Application Priority Data
Jun. 1, 2022 (AU) 2022901500

(51) Int. Cl. B01D 63/04; B01D 65/02; B01D 69/02; B01D 69/08; C07K 1/34

(52) U.S. Cl. CPC B01D 63/04; B01D 65/02; B01D 69/02; B01D 69/08; C07K 1/34; B01D 2321/04; B01D 2321/12; B01D 2321/168; B01D 2325/02833; B01D 2325/02834

(57) ABSTRACT

A method (100) of filtering solids (41) from a solution (44) derived from blood plasma is disclosed. The method (100) comprises feeding (102) the solution (44) into a hollow fibre filter (12) at a feed rate, the hollow fibre filter (12) comprising a plurality of hollow fibres (38), each hollow fibre (38) comprising a membrane (36) defining an elongate hollow fibre channel (32). The method (100) further comprises filtering (104) the solution (44) using the hollow fibre filter (12) to produce a permeate (46) and a retentate (45), the permeate (46) passing through pores (37) of the membrane (36) at a trans-membrane pressure and the retentate (45) flowing from respective outlets of the elongate hollow fibre channels (32), wherein the permeate (46) has a reduced solids content with respect to the solution (44) fed into the hollow fibre filter (12).

### TECHNICAL FIELD

The present disclosure relates, generally, to a method of filtering solids from liquids, and, more particularly, to a method of filtering solids from a solution derived from blood plasma.

### BACKGROUND

Advances in the understanding of the function of blood plasma proteins and the deficiencies involved in a variety of blood disorders, combined with improvements in techniques for storage of the major protein components of human blood, have resulted in increased utilisation of specific sub-fractions of human blood, in particular the cellular components (erythrocytes, thrombocytes and leukocytes) and plasma protein fractions (albumins, fibrinogen and globulins including euglobulins, pseudoglobulins, alpha-globulins, beta-globulins and gamma globulins, such as immunoglobulin G (IgG)), rather than whole blood, for therapeutic purposes.

The plasma protein fraction of human blood, in particular, is of enormous value to the pharmaceutical industry in the production of therapeutics for the treatment of fibrinogenic, fibrinolytic and coagulation disorders and immunodeficiencies, for example haemophilia, von Willebrand's disease and fibrinogen deficiency, amongst others.

Blood plasma fractions are formed from blood plasma fractionation processes such as the Cohn process, the Kistler and Nitschmann process or variations of these processes. These industrial scaled cold ethanol fractionation methods enable multiple plasma proteins to be extracted from the one plasma source. Such processes generally involve frozen plasma (batch sizes in the range of 1000-15000 kg) being thawed to form an albumin rich cryosupernatant and a cryoprecipitate. The cryoprecipitate contains valuable coagulation factors that are subsequently separated from the cryosupernatant. In the Cohn or Kistler and Nitschmann processes, the cryosupernatant may be optionally exposed to an initial low ethanol (typically 8%) precipitation stage to remove Fibrinogen. Again, the precipitate (Fraction I) is removed and can be used to make other products such as Fibrinogen. Adsorption steps using ion-exchange or affinity resins are also optionally conducted across either of these two intermediate fractions to extract other proteins (e.g. Prothrombin complex; Antithrombin III; C1 esterase inhibitor). Subsequently, the albumin is extracted from the Supernatant I by raising the ethanol concentration to about 25% at about pH 6.9 for the Cohn method or about 19% at about pH 5.85 for the Kistler and Nitschmann method, the immunoglobulins are precipitated (Fraction (I+)II+III or Precipitate A) while the albumin remains in solution (Supernatant (I+)II+III or Filtrate A). Albumin is then isolated from the majority of the other plasma contaminants (mainly a and ß globulins), which are precipitated by the further addition of ethanol to a final ethanol concentration of about 40% (Fraction IV). In a final step, the albumin is itself precipitated near its isoelectric point. The precipitate paste (Fraction V) can be held frozen before further processing. It is important to recognise that these processes have some adaptability and have been optimised over the years to suit each manufacturer's product portfolio. An example of this would be the presence or absence of an additional Cohn fractionation step (Fraction IV-1) following Fraction (I+)II+II step that can be used to extract alpha-1-antitrypsin. Another example is the use of Fraction IV-4 derived from Fraction II+III or Fraction I+II+III to extract hemopexin.

Separation processes are required to purify or separate larger solid contaminants from blood plasma fractions in order to further process blood plasma fractions to obtain the required protein component. A filter press is commonly used to separate the solid and liquid phases. The filtration process takes place in the filter plates of the filter press, where several filter plates are joined to one another to form a filter plate assembly. With the aid of a closing cylinder, usually a hydraulic closing cylinder, pressure is applied to the filter plate assembly in order to guarantee the necessary leak-tightness between the individual filter plates. Each of the filter plates has a filter area covered with a filter media, where the resuspended plasma fraction to be filtered is pressed into the filter chamber formed between two filter plates and against the filter media. Application of the product to the filter press allows for the liquid to pass through the filter media, whilst the solids are retained on the filter media. The filtrate between the filter media and the filter area is then carried off, and the filter cake remains in the filter chamber. The solids, which form a filter cake, are harvested from the filter press when the filter plates are separated from one another.

However, the operation of the filter press is labour intensive and time consuming. Further, filter presses have a large footprint and therefore require large spaces for storage and operation.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

In an aspect of the present disclosure, there is provided a method of filtering solids from a solution derived from blood plasma, the method comprising: feeding the solution into a hollow fibre filter at a feed rate, the hollow fibre filter comprising a plurality of hollow fibres, each hollow fibre comprising a membrane defining an elongate hollow fibre channel; and filtering the solution using the hollow fibre filter to produce a permeate and a retentate, the permeate passing through the pores of the membrane at a trans-membrane pressure and the retentate flowing from respective outlets of the elongate hollow fibre channels, wherein the permeate has a reduced solids content with respect to the solution fed into the hollow fibre filter.

In some embodiments, the method may further comprise recycling the retentate into the solution for feeding into the hollow fibre filter.

The feed rate of the solution can be defined by the cross flow velocity of the solution, i.e. the linear velocity of the flow tangential to the hollow fibre membrane surface. For example, the cross flow velocity may be from about 0.6 m/s to about 4 m/s. The pores of the membrane may have an average pore size of from about 0.1 microns to about 4 micron. The trans-membrane pressure may be from about 20 kilopascals to about 300 kilopascals.

The method may further comprise measuring the permeate output of the system. This can assist in assessing the performance of the system and identifying when filter performance is reducing and allow for timely corrective actions. In some embodiments, the method may comprise measuring a permeate flow rate during filtering. In some embodiments, the method may comprise measuring a permeate flux during filtering. The permeate flux may be at least 3 litres per square metre of the hollow fibre filter per hour.

In some embodiments, the method comprises multiple filtering steps. In such embodiments, the method further comprises feeding a backwash solution through the hollow fibre filter between filtering steps. The backwash solution may comprise a buffer solution. Alternatively or additionally the backwash solution may comprise permeate. The volume of backwash solution fed to the hollow fibre filter between filtering steps is provided in a ratio to the permeate volume obtained during the filtering step prior to backwash of from 1:6 to 1:1.

The solution derived from blood plasma may have a conductivity of from about 2 mS/cm to about 40 mS/cm. The solution may be at a temperature of from about 4° C. to about 37° C.

In an embodiment, the solution derived from blood plasma may have a conductivity of from about 2 mS/cm to about 8 mS/cm when measured at room temperature. In another embodiment, the solution derived from blood plasma may have a conductivity of from about 8 mS/cm to about 15 mS/cm when measured at room temperature. In another embodiment, the solution derived from blood plasma may have a conductivity of from about 25 mS/cm to about 40 mS/cm when measured at room temperature.

The solution may comprise a blood plasma fraction. The solution may comprise a buffer. The buffer may comprise sodium acetate or a phosphate. The solution may have an extraction ratio of kilograms of blood plasma fraction to kilograms of buffer of from about 1:2 to about 1:10.

The solution may comprise hemopexin, albumin, or immunoglobulin G. The method may further comprise adding octanoic acid to the permeate for delipidating the permeate. The solution may comprise a filter aid. The solution may have a pH of between about 4 and about 9.

A recovery of the permeate may be at least 30%, at least 50%, at least 75%, or at least 90%. A turbidity of the permeate may be less than about 400 nephelometric turbidity units.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

Embodiments of the disclosure will now be described by way of example only with reference to the accompanying drawings in which:

FIG. 1a shows an embodiment of a filtration system comprising a hollow fibre filter, shown in a filtering configuration;

FIG. 1b shows the embodiment of Figure la, shown in a backwashing configuration;

FIG. 2 shows an exploded, perspective view of the hollow fibre filter shown in FIG. 1, having channels in the form of hollow fibers;

FIG. 3 shows a perspective view of a hollow fiber;

FIGS. 4A-4D show a schematic of pores of the hollow fiber shown in FIG. 3 and illustrate a fouling mechanism of the pores;

FIG. 5 shows a flowchart of an embodiment of a method of removing solids from blood plasma fractionation solution using the hollow fibre filter shown in FIG. 1 to produce a permeate;

FIG. 6 shows a flowchart of another embodiment of the method shown in FIG. 5;

FIG. 7 shows a graph of experimental results of permeate flow rate versus time under different TMP values from applying the method shown in FIG. 5 to the solution;

FIG. 8 shows a graph of experimental results of permeate mass versus time under different TMP values from applying the method shown in FIG. 5 to the solution;

FIG. 9 shows a graph of experimental results of permeate flow rate versus time under different TMP values from applying the method shown in FIG. 5 to the solution;

FIG. 10 shows a graph of experimental results of permeate mass versus time under different TMP values from applying the method shown in FIG. 5 to the solution;

FIG. 11 shows a graph of experimental results of permeate mass post backwash versus time under different TMP values and pump speeds from applying the method shown in FIG. 6 to the solution;

FIG. 12 shows a graph of protein transmission versus accumulated permeate volume under different TMP values and pump speeds from applying the method shown in FIG. 6 to the solution;

FIG. 13 shows a graph of experimental results of average permeate flow rate versus accumulated backwash volume under different backwashing frequencies from applying the method shown in FIG. 6 to the solution;

FIG. 14 shows a graph of experimental results of average permeate flow rate versus time after each backwash for different quantities of backwashing cycles from applying the method shown in FIG. 6 to the solution;

FIG. 15 shows a column chart of experimental results of average permeate flow rate versus quantity of backwashing cycles from applying the method shown in FIG. 6 to the solution;

FIG. 16 shows a graph of experimental results of average permeate flow rate versus time from applying the method shown in FIG. 6 to the solution;

FIG. 17 shows a graph of protein transmission versus volume of permeate fractions for albumin compared to hemopexin from applying the method shown in FIG. 5 to the solution;

FIG. 18 shows a graph of experimental results of permeate flow rate versus time for different quantities of backwashing cycles from applying the method shown in FIG. 6 to the solution;

FIG. 19 shows a graph of experimental results of steady-state permeate flow rate versus quantity of backwashing cycles from applying the method shown in FIG. 6 to the solution;

FIG. 20 shows a graph of experimental results of permeate mass versus time under different TMP increase rates from applying the method shown in FIG. 5 to the solution;

FIG. 21 shows a graph of protein transmission versus volume of permeate fractions for hemopexin extract under different pH levels of the solution from applying the method shown in FIG. 5 to the solution;

FIG. 22 shows a graph of permeate flow rate versus time under different TMP values compared to the theoretical cake formation fouling model;

FIG. 23A shows a graph of permeate flow rate versus time compared to the theoretical partial blocking, internal blocking and cake formation fouling models at an early stage of the experiment;

FIG. 23B shows a graph of permeate flow rate versus time compared to the theoretical partial blocking, internal blocking and cake formation fouling models at a late stage of the experiment; and

FIG. 24 shows a graph of permeate flow rate versus time compared to the theoretical partial blocking and cake formation fouling models.

### DETAILED DESCRIPTION OF EXEMPLARY EMBODIMENTS

### General Terms

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects unless the context clearly dictates otherwise. For example, reference to “a” includes a single as well as two or more; reference to “an” includes a single as well as two or more; reference to “the” includes a single as well as two or more and so forth.

Each example of the present disclosure described herein is to be applied mutatis mutandis to each and every other example unless specifically stated otherwise. The present disclosure is not to be limited in scope by the specific examples described herein, which are intended for the purpose of exemplification only. Functionally-equivalent products, compositions and methods are clearly within the scope of the disclosure as described herein.

The term “and/or”, e.g., “X and/or Y” shall be understood to mean either “X and Y” or “X or Y” and shall be taken to provide explicit support for both meanings or for either meaning.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting.

The term “about” as used herein means within 5%, and more preferably within 1%, of a given value or range. For example, “about 3.7%” means from 3.5 to 3.9%, preferably from 3.66 to 3.74%. When the term “about” is associated with a range of values, e.g., “about X % to Y %”, the term “about” is intended to modify both the lower (X) and upper (Y) values of the recited range. For example, “about 20% to 40%” is equivalent to “about 20% to about 40%”.

### Specific Terms

The term “recovery” or “recovery percentage” is used to refer to the concentration of protein, for example, hemopexin, albumin, IgG or any other desirable protein, in the permeate 46 as a percentage of the maximum possible concentration of the protein in the permeate 46, taking into account the mass difference between the permeate 46 and the solution 44. The concentrations may be measured using Reversed-phase High Performance Liquid Chromatography (RP-HPLC) or nephelometry or ultra-violet spectroscopy, for example. The maximum possible concentration of the protein in the permeate 46 can be calculated using the following formula:

$${C}_{p , max} = \frac{{m}_{e}}{{m}_{p}} \times {C}_{e}$$

Where: C_{p,max} is the maximum concentration of the protein in the permeate 46 (g/L)
mₑ is the mass of the extract or solution 44 (kg)
mₚ is the mass of the final permeate (kg)
Cₑ is the concentration of the extract or solution 44 (g/L)

Recovery may be used as a performance indicator with the operating parameters of the hollow fibre filter 12 being varied to achieve a desired recovery. It will be appreciated that the desired recovery will vary based on a variety of factors, for example the composition of the feed solution 44, the quantities of backwashing cycles. The properties and/or operating conditions of the hollow fibre filter 12 and the inclusion of one or more further separation/filtration processes may be selected to provide a desired recovery and clarity.

The term “permeate flow rate” is defined by the mass of the permeate 46 flowing out of the hollow fibre filter 12 through the permeate line 24 per unit of time and is represented by grams per minute (g/min). It will be understood that any suitable units may be used such as pounds of the permeate per hour. Permeate flow rate may be used as a performance indicator of the method 100, with a higher permeate flow rate being typically indicative of improved productivity and filter performance (e.g. the filter not being clogged, fouled or obstructed by solids). The properties and/or operating conditions of the hollow fibre filter 12 and properties of the solution 44 may be selected to provide a desired permeate flow rate.

The term “permeate flux” of “flux” is defined by the total volume of the permeate 46 filtered per unit surface area of the hollow fibre filter 12 per unit of time, represented by litres of the permeate 46 per square metre of the filter per hour (L/m2/h). It will be understood that any suitable units may be used such as gallons of the permeate 46 per square foot of the filter per second. Flux may be used as a performance indicator of the method 100, with higher flux typically indicative of improved productivity and filter performance (e.g. the filter not being clogged/fouled or obstructed by solids). As with the permeate flowrate, properties and/or operating conditions of the hollow fibre filter 12 and properties of the solution 44 may be selected to provide a desired flux.

The term “complexed hemopexin” or “complexed hemopexin percentage” is defined by the percentage weight of hemopexin in the solution 44, the retentate 45, or the permeate 46, that is bound to heme, rather than being freely available as hemopexin. This may be measured using Size Exclusion-High Performance Liquid Chromatography (SEC-HPLC), for example.

The term “protein transmission” or “protein transmission percentage” is defined by the concentration of the protein, for example, hemopexin, albumin, IgG or any other desirable protein, in the permeate 46 as a percentage of the concentration of that protein in the retentate 45, which may be determined by protein A280, for example.

### Method of Removing Solids From A Solution Derived from Blood Plasma

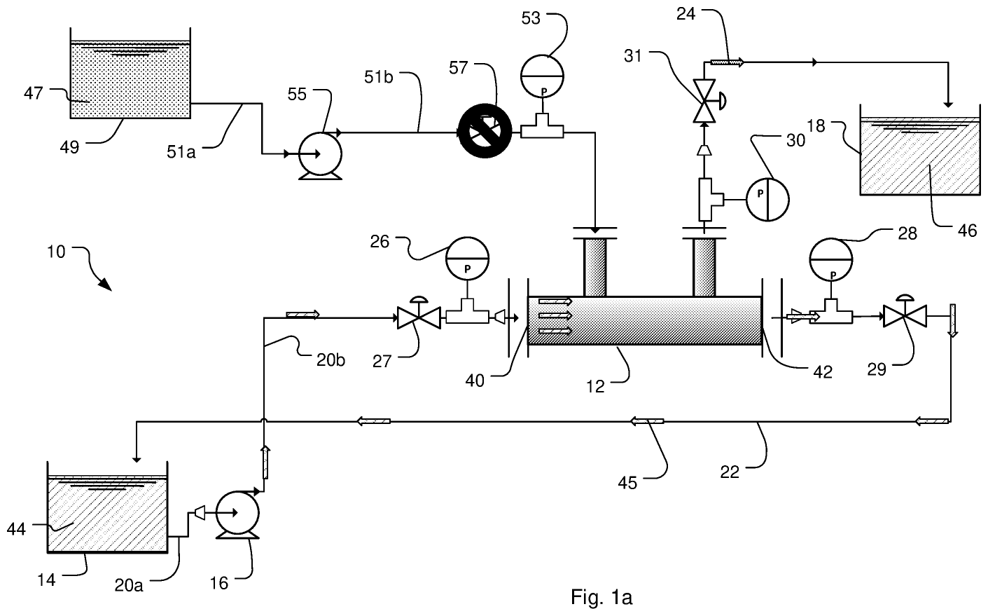

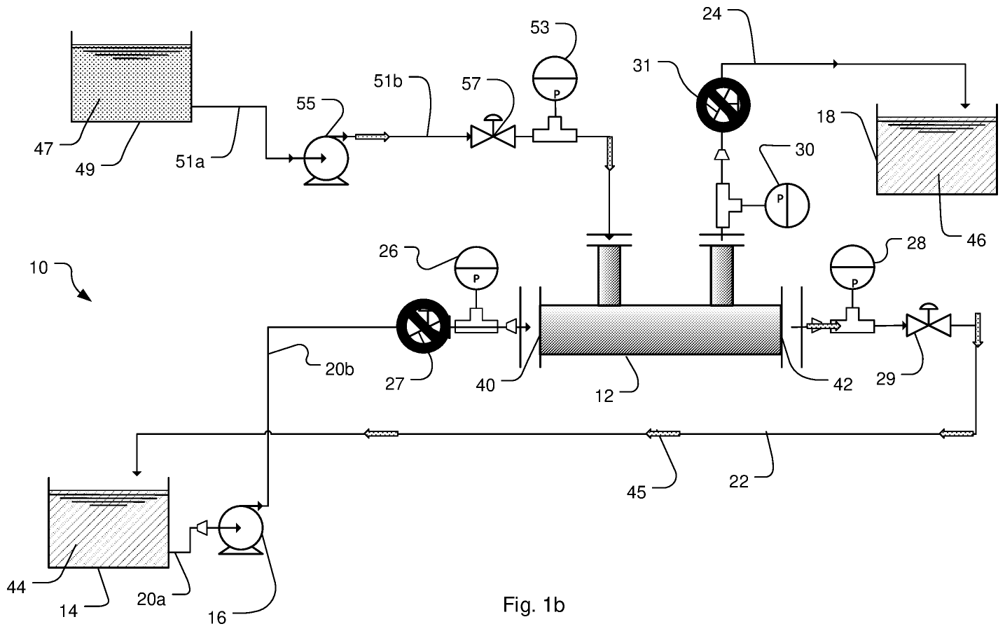

Referring initially to FIGS. 1a and 1b, there is shown a filtration system 10 comprising a hollow fibre filter 12. A feed tank 14 is connected to an inlet of the hollow fibre filter 12 via feedlines 20a, 20b, a feed pump 16, and feed valve 27. A retentate outlet of the hollow fibre filter 12 is connected to the feed tank 14 via retentate line 22 and retentate valve 29, and a permeate outlet of the hollow fibre filter 12 is connected to a permeate tank 18 via permeate line 24 and permeate valve 31. A feed pressure gauge 26, retentate pressure gauge 28, and permeate pressure gauge 30 are provided on the feed lines 20a, 20b, retentate line 22 and permeate line 24 respectively. A backwash circuit is also provided comprising a backwash tank 49, backwash lines 51a, 51b, a backwash valve 57, a backwash pump 55, and a backwash pressure gauge 53. In an alternate embodiment, a fluid feedline (not shown) connecting the permeate tank 18 and the backwash circuit may be provided.

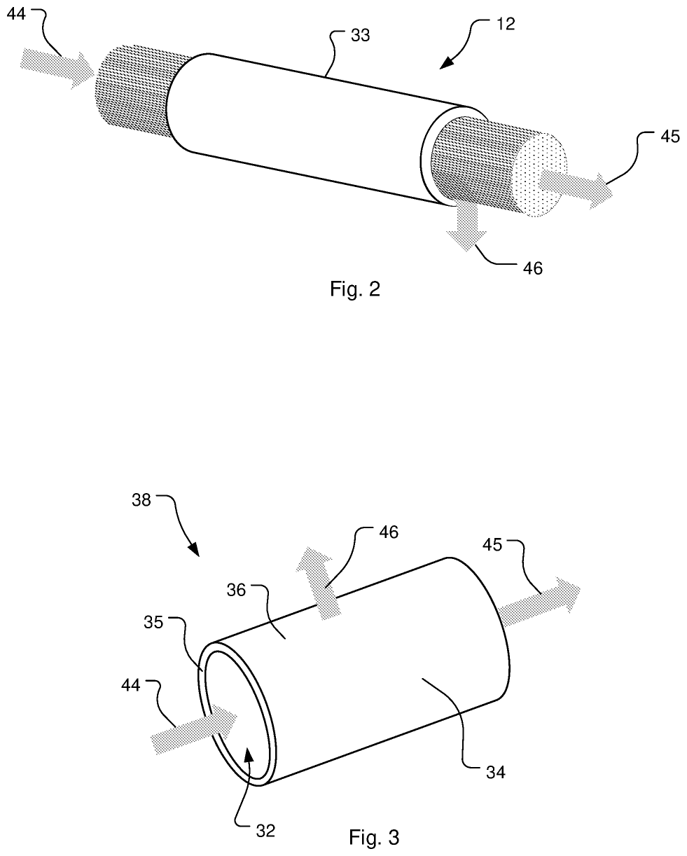

An embodiment of a hollow fibre filter 12 is shown in FIGS. 2. The hollow fibre filter 12 comprises a filter housing 33 and a plurality of hollow fibres 38 positioned within the housing. As best shown in FIG. 3, each hollow fibre 38 comprises a membrane 36 defining an elongate hollow fibre channel 32. The hollow fibres may be any suitable hollow fibre for performing solid/liquid separation. By way of example, with reference to FIG. 3, the hollow fibres may comprise a porous support 35 and a wall 34 around the porous support 35. The wall 34 comprises a membrane 36 with pores 37 characterised by their pore size. The hollow fibres 38 are secured to end caps 40, 42 of the hollow fibre filter 12 (FIG. 1). The hollow fibres 38 may be formed from any suitable material, for example the hollow fibres 38 may be formed of ceramic or polymeric materials. In an embodiment, the hollow fibres 38 are formed from silicon carbide. It will be understood that the channels 32 of the hollow fibre filter 12 may instead be formed as through holes in a porous material which acts as the membrane 36, instead of having discrete hollow fibres 38.

The total filter area for the hollow fibre filter 12 is defined by the sum of the area of the membranes 36 of all of the hollow fibres 38. Although primarily described with reference to a single filtration unit, it will be appreciated that the described system may include multiple individual hollow fibre filtration units in parallel and/or in series, in particular to increase the scale of the operation and volume of solution able to be filtered.

In an embodiment, the method may be performed using an individual hollow fibre filter unit. The filter area of an individual hollow fibre filter unit according to the present disclosure may be from about 0.1 m² to about 10 m². For example, the filter area of an individual unit may be about 0.05 m², about 0.1 m², about 0.15 m², about 0.2 m², about 0.25 m², about 0.3 m², about 0.35 m², about 0.4 m², about 0.45 m², about 0.5 m², about 0.6 m², about 0.7 m², about 0.8 m², about 0.9 m², about 1 m², about 1.1 m², about 1.2 m², about 1.3 m², about 1.4 m², about 1.5 m², about 1.6 m², about 1.7 m², about 1.8 m², about 1.9 m², about 2.0 m², about 2.5 m², about 3.0 m², about 3.5 m², about 4.0 m², about 4.5 m², about 5.0 m², about 5.5 m², about 6.0 m², about 6.5 m², about 7.0 m², about 7.5 m², about 8.0 m², about 8.5 m², about 9.0 m², about 9.5 m², or about 10.0 m². The filter area of an individual hollow fibre filter unit may be in a range any two of the above listed filter areas.

In some embodiments, the method may be performed using two or more individual hollow fibre filter units in operated in parallel. Each individual hollow fibre filter unit may have a filter area as defined above. The individual hollow fibre filter units operated in parallel may each have the same filter area, or the filter area may vary for some or all of the individual filter units. It will be appreciated that the total filter area for the system will be defined by the sum of the filter areas for each of the individual units.

The length of the hollow fibres 38 may be any suitable length. Typically, although not necessarily so, the channels 32 have a substantially circular cross section. The diameter of the channels 32 may be from about 2 mm to about 20 mm, for example about 2 mm, about 3 mm, about 4 mm, about 5 mm, about 6 mm, about 7 mm, about 8 mm, about 9 mm, about 10 mm, about 11 mm, about 12 mm, about 13 mm, about 14 mm, about 15 mm, about 16 mm, about 17 mm, about 18 mm, about 19 mm, or about 20 mm. In an embodiment, the diameter is 3.2 mm. It will be understood that the channels 32 may have other cross-section shapes, for example elliptical or polygonal.

During filtering, the feed valve 27, retentate valve 29 and permeate valve 31 are open and a solution 44 is pumped from the feed tank 14 using feed pump 16 into the the elongate hollow fibre channels 32 via endcap 40 of the hollow fibre filter 12. During filtering, the backwash valve 57 is closed. Particles in the solution 44 that are smaller than the pore size of the hollow fibre membrane 36 are pushed through the hollow fibre membrane 36, driven by the trans-membrane pressure (TMP), and flow through the permeate line 24 as a permeate 46 for collection in the permeate tank 18. The remaining portion of the solution, the retentate 45, flows through the elongate hollow fibre channels 32 to the retentate line 22 for recycling 105 to the feed tank 14. The recirculation of the retentate 45 and filtering of the solution 44 may continue until the desired volume or recovery of the permeate 46 is reached.

As filtration progresses, fouling of the hollow fibres 38 may occur whereby pores 37 of the membranes 36 become clogged with solids 41, which in turn may reduce the efficiency of the filter. This may be indicated by a reduction in the permeate flow rate or permeate flux, or a change in the trans-membrane pressure (TMP).

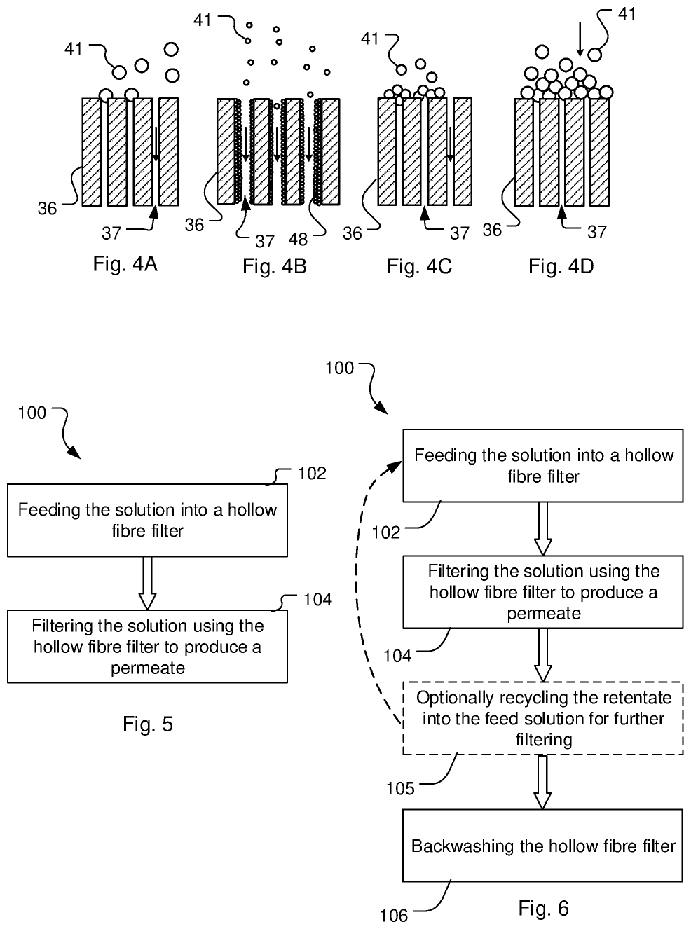

The four main mechanisms of fouling or clogging include the complete pore blocking model in which the solid particles completely cover and block the pores 37 (FIG. 4A), the standard/internal blocking model in which the solids 41 gather on an internal wall 48 of the pores 37 (FIG. 4B), the intermediate/partial blocking model in which particles accumulate on the membrane surface and block some of the pores 37 (FIG. 4C), and the cake filtration/formation model in which particles accumulate and cover the membrane surface forming a cake with low permeability on the hollow fibre membrane 36 (FIG. 4D). These models are defined by the following formulas [Brião, V. B., Seguenka, B., Zanon, C. D., & Milani, A. (2017). Cake formation and the decreased performance of whey ultrafiltration. Acta Scientiarum. Technology, 39(5), 517-524]:

| Pore blocking | | | Equation |
|---|---|---|---|
| mechanism | Final Equation | Constant | Number |
| Complete pore blocking | $J = {J}^{*} + ( {J}_{0} - {J}^{*} ) {e}^{( - {k}_{α} \cdot t )}$ | ${k}_{α} = \frac{{J}_{0} \cdot σ}{{ε}_{0}}$ | (1) |
| Internal pore blocking | $\frac{1}{{J}^{1 / 2}} = \frac{1}{{J}_{0}^{1 / 2}} + {k}_{b} \cdot t$ | ${k}_{b} = \frac{{k}_{1}}{2} \cdot {A}^{1 / 2}$ | (2) |
| Intermediate pore blocking | ${k}_{c} \cdot t = \frac{1}{{J}^{*}} ln ( \frac{J}{{J}_{0}} \cdot \frac{( {J}_{0} - {J}^{*} )}{( J - {J}^{*} )} ) .$ | k_{c} = σ | (3) |
| Cake formation | ${k}_{d} \cdot t = \frac{1}{{J}^{* 2}} [ ln ( \frac{J}{{J}_{0}} \cdot \frac{( {J}_{0} - {J}^{*} )}{( J - {J}^{*} )} ) ] - {J}^{*} ( \frac{1}{J} - \frac{1}{{J}_{0}} )$ | ${k}_{d} = \frac{α \cdot {k}_{t}}{{J}_{0} {R}_{m}}$ | (4) |

Where: kₐ, k_{b}, k_{c}, and k_{d} are constants of the models
J* is the critical flow rate which should not be exceeded to avoid fouling
J is the permeate flow rate after time t
J₀ is the initial permeate flow rate
ε₀ is the membrane surface porosity of the cleaned membrane 36
A is the membrane surface area
σ is the blocked membrane area per unit permeate volume
α is a parameter characterising fouling potential of the solution
Rₘ is the cleaned membrane resistance

The filtration process may be periodically paused or the feed rate may be temperately reduced to allow for backwashing of the hollow fibre filter to remove of fouling and unclog pores by pushing a backwash solution 47 such as a buffer and/or the permeate through the filter 12 from the permeate side to clean the fouled membrane 36.

Referring to FIG. 5, there is provided an embodiment of a method 100 of filtering solids 41 from a solution 44 derived from blood plasma. The method 100 comprises feeding 102 the solution 44 into the hollow fibre filter 12 at a feed rate, the hollow fibre filter 12 comprising a plurality of hollow fibres 38, each hollow fibre 38 comprising a membrane 36 defining an elongate hollow fibre channel 32. The method 100 further comprises filtering 104 the solution 44 using the hollow fibre filter 12 to produce a permeate 46 and a retentate 45, the permeate 46 passing through the pores 37 of the membrane 36 at a trans-membrane pressure (TMP) and the retentate 45 flowing from respective outlets of the elongate hollow fibre channels 32, wherein the permeate 46 has a reduced solids content with respect to the solution 44 fed into the hollow fibre filter 12.

The step of feeding 102 the solution may be performed using the pump 16, which may be for example a centrifugal pump. The feed rate of the solution 44 may be defined by a cross flow velocity, where the volumetric flow rate is a function of the cross flow velocity and the cross sectional area of the fibre channels. The cross flow velocity of the feed may be from about 0.6 m/s to about 4.0 m/s. In some embodiments the cross flow velocity may be about 0.6 m/s, about 0.7 m/s, about 0.8 m/s, about 0.9 m/s, about 1.0 m/s, about 1.1 m/s, about 1.2 m/s, about 1.3 m/s, about 1.4 m/s, about 1.5 m/s, about 1.6 m/s, about 1.7 m/s, about 1.8 m/s, about 1.9 m/s, about 2.0 m/s, about 2.2 m/s, about 2.4 m/s, about 2.6 m/s, about 2.8 m/s, about 3.0 m/s, about 3.2 m/s, about 3.4 m/s, about 3.6 m/s, 3.8 m/s, or about 4.0 m/s.

The feed rates are related to pump speed via the formula:

$${V}_{1} {n}_{2} = {V}_{2} {n}_{1}$$

Where: V₁, V₂ are two different feed rates (L/min)
n₁, n₂are the two corresponding pump speeds (rpm)

The pump speeds may be set on the pump 16 using either rpm or hertz (Hz) units which can be converted to rpm by multiplying by 60.

The average pore size 39 may be between about 0.1 microns and about 4 microns, for example, from about 0.2 microns to about 1 micron. In some embodiments, the pore size 39 may be about 0.05 microns, about 0.1 microns, about 0.15 microns, about 0.2 microns, about 0.25 microns, about 0.3 microns, about 0.35 microns, about 0.4 microns, about 0.45 microns, about 0.5 microns, about 0.55 microns, about 0.6 microns, about 0.65 microns, about 0.7 microns, about 0.75 microns, about 0.8 microns, about 0.85 microns, about 0.9 microns, about 0.95 microns, about 1 micron, about 1.25 micron, about 1.5 micron, about 1.75 micron, about 2 micron, about 2.5 micron, about 3 micron, about 3.5 micron, or about 4 micron. It will be appreciated that the desired pore size may vary depending on the properties of the solution to be treated and the desired final properties of the permeate.

It will be understood that the pores 37 may be distributed in a homogenous, even or substantially even manner across the membrane 36, such that the distance between the pores 37 is substantially equal. It will also be understood that the pores 37 may be distributed non-homogenously or unevenly such that the distance between some pores 37 is less or more than the distance between other pores 37.

The TMP is defined by the following formula:

$$Δ P = \frac{{P}_{i} + {P}_{o}}{2} - {P}_{p}$$

Where: ΔP is the TMP (kPa)
Pᵢ is the inlet pressure (kPa), e.g. the feed pressure gauge 26
Pₒ is the outlet pressure (kPa), e.g. the retentate pressure gauge 28
Pₚ is the permeate pressure (kPa), e.g. the permeate pressure gauge 30

The TMP may be varied by adjusting one or more of the feed valve, the retentate valve, and/or the permeate valve, in combination with the other operating parameters that may alter the inlet pressure and/or outlet pressure. The TMP may be between about 10 kPa and about 300 kPa, for example, between about 20 kPa and 300 kPa. In some embodiments, the TMP may be set to about 10 kPa, about 20 kPa, about 25 kPa, about 30 kPa, about 35 kPa, about 40 kPa, about 45 kPa, about 50 kPa, about 55 kPa, about 60 kPa, about 65 kPa, about 70 kPa, about 75 kPa, about 80 kPa, about 85 kPa, about 90 kPa, about 95 kPa, about 100 kPa, about 105 kPa, about 110 kPa, about 115 kPa, about 120 kPa, about 125 kPa, about 130 kPa, about 135 kPa, about 140 kPa, about 145 kPa, about 150 kPa, about 160 kPa, about 170 kPa, about 180 kPa, about 190 kPa, about 200 kPa, about 210 kPa, about 220 kPa, about 230 kPa, about 240 kPa, about 250 kPa, about 260 kPa, about 270 kPa, about 280 kPa, about 290 kPa, or about 300 kPa. The TMP may be set in a range between any two of the above listed pressures.

In some embodiments, the solution 44 comprises a blood plasma fraction, such as Fraction (I+)II+III comprising IgG, Fraction IV-4 comprising hemopexin, and Fraction V comprising albumin. The person skilled in the art will understand that the solution 44 is not limited to the use of blood plasma fractions, and that the blood plasma fractions are not limited to the aforementioned fractions and may include any suitable plasma fraction, such as any of those stated in the background section. The person skilled in the art will also understand that the solution 44 may be derived from other sources of blood such as animal blood, for example, bovine blood.

The use of different blood plasma fractions may require selecting different operating parameters of the hollow fibre filter 12 in order to obtain a higher recovery, a higher permeate flow rate, a higher permeate flux and/or a lower turbidity of the permeate 46. Also, certain blood plasma fractions may provide a higher recovery and a lower turbidity than other blood plasma fractions under the same operating parameters. For example, the use of Fraction IV-4 comprising hemopexin may provide a higher recovery and a lower turbidity as seen in the examples section.

The temperature of the solution 44 may be any suitable temperature for the composition of the solution. In an embodiment, the solution is maintained at a temperature in the range of from about 4° C. to about 37° C. In some embodiments, the temperature of the solution is less than about 25° C.

Preferably, the conductivity of the solution is from about 2 mS/cm to about 40 mS/cm. For example, the conductivity of the solution may be about 2 mS/cm, 3 mS/cm, 4 mS/cm, 5 mS/cm, 6 mS/cm, 7 mS/cm, 8 mS/cm, 9 mS/cm, 10 mS/cm, 11 mS/cm, 12 mS/cm, 13 mS/cm, 14 mS/cm, 15 mS/cm, 16 mS/cm, 17 mS/cm, 18 mS/cm, 19 mS/cm, 20 mS/cm, 21 mS/cm, 22 mS/cm, 23 mS/cm, 24 mS/cm, 25 mS/cm, 26 mS/cm, 27 mS/cm, 28 mS/cm, 29 mS/cm, 30 mS/cm, 31 mS/cm, 32 mS/cm, 33 mS/cm, 34 mS/cm, 35 mS/cm, 36 mS/cm, 37 mS/cm, 38 mS/cm, 39 mS/cm, or 40 mS/cm. While typically, the conductivity of the solution is measured at room temperature, it will be appreciated that the conductivity may also be measured at the given temperature of the solution being fed to the hollow fibre filter. In an embodiment, the solution derived from blood plasma may have a conductivity of from about 8 mS/cm to about 15 mS/cm when measured at room temperature.

The solution 44 may further comprise a buffer. In these embodiments, buffer is added to a product derived from blood plasma to form the solution 44 prior to being fed to the hollow fibre filter 12. The purpose of the buffer is to resuspend the blood plasma fraction, which is a precipitate and may be in the form of a paste, so that the solution 44 has a suitable viscosity to be fed into the hollow fibre filter 12. Any suitable buffer may be used, for example the buffer may comprise sodium acetate, water (e.g. water for injection (WFI)), or a phosphate, such as disodium phosphate.

The conductivity of the solution may be adjusted as required to achieve the desired conductivity. Where the solution comprises a buffer solution, the concentration of the buffer may be adjusted to achieve the desired conductivity without substantial dilution of the sample.

The ratio of blood plasma fraction to buffer used in the solution 44 may affect the recovery, permeate flow rate and/or turbidity of the permeate 46. This ratio is termed an extraction ratio of the solution 44 and is defined by kilograms of the blood plasma fraction to kilograms of the buffer. It will be appreciated that the extraction ratio may be varied depending on the fraction of plasma being used as well as practical considerations such as the space required to store and use the volume of buffer required for higher extraction ratios. For example, extraction ratios of 1:1 or higher may be used, but for a higher recovery at least an extraction ratio of 1:2 would be suitable. A practical maximum extraction ratio would be 1:20, though it will be understood by the person skilled in the art that the extraction ratio may exceed 1:20 if required to achieve a particular recovery and/or to resuspend a particular blood plasma fraction.

Considering these factors, the solution 44 may have an extraction ratio of from about 1:2 to about 1:10. However, the extraction ratio may be any suitable extraction ratio, for example the extraction ratio may be about 1:1, about 1:1.5, about 1:2, about 1:2.5, about 1:3, about 1:3.5, about 1:4, about 1:4.5, about 1:5, about 1:5.5, about 1:6, about 1:6.5, about 1:7, about 1:7.5, about 1:8, about 1:8.5, about 1:9, about 1:9.5, about 1:10, about 1:10.5, about 1:11, about 1:11.5, about 1:12, about 1:12.5, about 1:13, about 1:13.5, about 1:14, about 1:14.5, about 1:15, about 1:15.5, about 1:16, about 1:16.5, about 1:17, about 1:17.5, about 1:18, about 1:18.5, about 1:19, about 1:19.5, about 1:20, or about 1:20.5.

In some embodiments, the solution 44 has a pH of between about 4 and about

9. For example, the solution 44 may have a pH of about 4, about 4.5, about 5, about 5.5, about 6, about 6.5, about 7, about 7.5, about 8, about 8.5, or about 9.

In some embodiments, the solution 44 comprises a filter aid. In these embodiments, the filter aid comes from blood plasma fractionation process. The filter aid may be any suitable filter aid, for example the filter aid may be cellulose based or silica based.

The method 100 may provide the permeate 46 with a recovery of at least 30%. In some embodiments, the method may provide a permeate 46 with a recovery of at least 30%, at least 50%, at least 75% or at least 90%.

Increasing recovery will allow for less loss of proteins, which is indicative of less product remaining in the retentate. Operating parameters such as feed rate, pore size, and/or trans-membrane pressure, as well as parameters of the solution 44, may be selected to increase recovery. It will be understood that the recovery of the permeate 46 may be at least about 20%, at least about 25%, at least about 30%, at least about 35%, at least about 40%, at least about 45%, at least about 50%, at least about 55%, 60%, at least about 62.5%, at least about 65%, at least about 67.5%, at least about 70%, at least about 72.5%, at least about 75%, at least about 77.5%, at least about 80%, at least about 82.5%, at least about 85%, at least about 87.5%, at least about 90%, at least about 92.5%, or at least about 95%.

The method 100 may be performed such that a turbidity of the permeate 46 is less than about 400 nephelometric turbidity units (NTU). In some embodiments, a lower turbidity of the permeate 46 can be a general indicator of increased removal of solids, therefore it is desirable for the permeate 46 to have a low turbidity. Operating parameters such as pore size and parameters of the solution 44, may be selected to lower turbidity. It will be understood that the turbidity of the permeate 46 may be less than about 600 NTU, less than about 550 NTU, less than about 500 NTU, less than about 450 NTU, less than about 400 NTU, less than about 375 NTU, less than about 350 NTU, less than about 325 NTU, less than about 300 NTU, less than about 275 NTU, less than about 250 NTU, less than about 225 NTU, less than about 200 NTU, less than about 175 NTU, less than about 150 NTU, less than about 125 NTU, less than about 100 NTU, less than about 75 NTU, less than about 50 NTU, or less than about 25 NTU.

In some embodiments, for example where the solution 44 comprises IgG, the method 100 further comprises adding octanoic acid to the solution 44 for delipidating the solution 44. In other embodiments, octanoic acid is added to the permeate 46 for delipidating the permeate 46. Where octanoic acid is used, calcium phosphate may be added to the solution 44 or the permeate 46 to neutralise any excess octanoic acid.

As shown for example in FIG. 6, the method 100 may further comprise backwashing 106 the hollow fibre filter 12. The backwashing 106 comprises closing the permeate valve 31, with backwash valve 57, feed valve 27, and retentate valve 29 open. A backwash solution 47 such as a buffer, preferably the same buffer as used in the solution 44, is pumped using the backwash pump 55 through buffer lines 51a, 51b into the permeate side of the hollow fibre filter 12, flushing the pores 37 of the membrane 38 to remove fouling and blockages. The backwash solution 47 with the solids flushed from membrane flows through the longitudinal channels 32 and through the retentate line 22. This solution of the backwash solution 47 and flushed solids are recycled to the feed tank for further filtration.

Backwashing 106 may be performed before the feeding 102 and/or after the filtering 104. The backwashing may be performed at a certain interval using a certain volume of backwash solution 47 for the backwashing cycle, for example, at least at a rate of 500 mL of backwash solution 47 per 2 L of permeate 46 produced. In some embodiments, the volume of backwash solution per volume of permeate may be about 100 mL per 2 L, about 150 mL per 2 L, about 200 mL per 2 L, about 250 mL per 2 L, about 300 mL per 2 L, about 350 mL per 2 L, about 400 mL per 2 L, about 450 mL per 2 L, about 500 mL per 2 L, about 550 mL per 2 L, about 600 mL per 2 L, about 650 mL per 2 L, about 700 mL per 2 L, about 750 mL per 2 L, about 800 mL per 2 L, about 850 mL per 2 L, about 900 mL per 2 L, about 950 mL per 2 L, about 1 L per 2 L, about 1.1 L per 2 L, about 1.2 L per 2 L, about 1.3 L per 2 L, about 1.4 L per 2 L, about 1.5 L per 2 L, about 1.6 L per 2 L, about 1.7 L per 2 L, about 1.8 L per 2 L, about 1.9 L per 2 L, about 2 L per 2 L, about 2.5 L per 2 L, about 3 L per 2 L, about 100 mL per L, about 150 mL per L, about 200 mL per L, about 250 mL per L, about 300 mL per L, about 350 mL per L, about 400 mL per L, about 450 mL per L, about 500 mL per L, about 550 mL per L, about 600 mL per L, about 650 mL per L, about 700 mL per L, about 750 mL per L, about 800 mL per L, about 850 mL per L, about 900 mL per L, about 950 mL per L, about 1 L per L, about 1.1 L per 1 L, about 1.2 L per L, about 1.3 L per L, about 1.4 L per L, about 1.5 L per L, about 1.6 L per L, about 1.7 L per L, about 1.8 L per L, about 1.9 L per L, about 2 L per L, about 2.5 L per L, or about 3 L per L.

In some embodiments, at least two backwashing cycles are performed during the method 100. In some embodiments, the number of backwashing cycles performed may be 1, 2,3, 4, 5, 6, 7, 8, 9, 10, 11, 12, 13, 14, 15, 16, 17, 18, 19, 20, about 25, about 30, about 35, about 40, about 45, about 50, or greater. In some embodiments, the method may be a continuous process, continuously cycling between filtering and backwashing.

Where the backwashing 106 is included in the method 100, operating parameters of the backwashing, such as backwashing interval and number of backwashing cycles, may be selected in addition to operating parameters of the hollow fiber filter 12 such as feed rate, pore size and/or TMP, as well as parameters of the solution 44, to achieve a desired recovery and/or turbidity of the permeate 46. The recovery and/or turbidity of the permeate 46 may be assessed after one or more backwashing cycles.

In some embodiments, the method further comprises measuring a permeate flow rate during filtering. Operating parameters such as feed rate, pore size and/or TMP and parameters of the solution 44, as well as backwashing interval and/or number of backwashing cycles, may be selected to achieve a desired permeate flow rate.

In some embodiments, the method further comprises measuring a permeate flux during filtering. Operating parameters such as feed rate, pore size and/or TMP and parameters of the solution 44, as well as backwashing interval and/or number of backwashing cycles, may be selected to achieve a desired permeate flux.

In some embodiments, the permeate flux may be about 0.5 L/m²/h, about 1 L/m²/h, about 2 L/m²/h, about 2.5 L/m²/h, about 3 L/m²/h, about 3.5 L/m²/h, about 4 L/m²/h, about 4.5 L/m²/h, about 5 L/m²/h, about 5.5 L/m²/h, about 6 L/m²/h, about 6.5 L/m²/h, about 7 L/m²/h, about 7.5 L/m²/h, about 8 L/m²/h, about 8.5 L/m²/h, about 9 L/m²/h, about 9.5 L/m²/h, about 10 L/m²/h, about 10.5 L/m²/h, about 11 L/m²/h, about 11.5 L/m²/h, about 12 L/m²/h, about 12.5 L/m²/h, about 13 L/m²/h, about 13.5 L/m²/h, about 14 L/m²/h, about 14.5 L/m²/h, about 15 L/m²/h, about 15.5 L/m²/h, about 16 L/m²/h, about 16.5 L/m²/h, about 17 L/m²/h, about 17.5 L/m²/h, about 18 L/m²/h, about 18.5 L/m²/h, about 19 L/m²/h, about 19.5 L/m²/h, about 20 L/m²/h, about 21 L/m²/h, about 22 L/m²/h, about 23 L/m²/h, about 24 L/m²/h, about 25 L/m²/h, about 30 L/m²/h, about 35 L/m²/h, or about 40 L/m²/h, about 50 L/m²/h, about 60 L/m²/h, about 70 L/m²/h, about 80 L/m²/h, about 90 L/m²/h, about 100 L/m²/h, about 150 L/m²/h, about 200 L/m²/h, about 250 L/m²/h, about 300 L/m²/h, about 350 L/m²/h, about 400 L/m²/h, about 450 L/m²/h, about 500 L/m²/h, about 550 L/m²/h, or about 600 L/m²/h. The permeate flux may be in a range between any two of the above values.

The permeate flow rate and/or permeate flux may be monitored to provide an indication of the filter performance over time. Once filtering of the solution is initiated, the permeate flow rate will quickly achieve a steady state, for example at one of the flow rates set out above. As filtering progresses and more fouling of the pores occurs, the permeate flow rate may decline from the steady state permeate flow rate. Once the permeate flow rate has reduced below a threshold flow rate value, which may be a percentage of the steady state flow rate, the filtering may be stopped and a backwashing step performed. The flux may be similarly monitored to assess filter performance.

The method 100 may further comprise additional filtering of the permeate, for example using a depth filter, a membrane filter, or a combination thereof. However, it will be understood by the person skilled in the art that other filtering mediums and techniques may be used alternatively or in addition to the depth filter and/or the membrane filter, such as filter papers, glass microfiber filters, prefilters, and the like. It will also be understood that the permeate or the filtered permeate using the depth filter and/or the membrane filter constitutes a blood plasma product produced using the method 100.

Where additional filtering is included in the method 100, properties and operation of the filters, such as filter type, filter pore size and/or retention rating, may be selected in addition to operating parameters of the hollow fibre filter 12, as well as parameters of the solution 46, to achieve a desired recovery and/or turbidity of the filtered permeate.

Advantageously, the use of the hollow fibre filter 12 in the method 100 removes solids from the solution 44 to an extent comparable to existing techniques such as a filter press, while providing a reduced footprint and a faster overall process. It will be appreciated that operation of the hollow fibre filter 12 is also less labour intensive than the filter press and does not require the emptying of individual filter plates which adds to the total processing time.

In some embodiments, the use of the hollow fibre filter 12 in the method 100 may provide a recovery of at least 60%, for example at least 75% or even at least 90%, thereby providing a comparable or better recovery to the use of a filter press. In some embodiments of the method 100, the permeate flux may be at least 30 L/m²/h, which is a comparable value to that of the filter press, thus indicating that the method 100 may be as effective and fast as the filtration using the filter press, while providing the advantages of a reduced footprint and labour as stated above.

It will be understood that the permeate 46 using the hollow fibre filter 12 constitutes a blood plasma product produced using the method 100.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### EXAMPLES

The following examples are to be understood as illustrative only. The following examples should therefore not be construed as limiting the embodiments of the disclosure in any way.

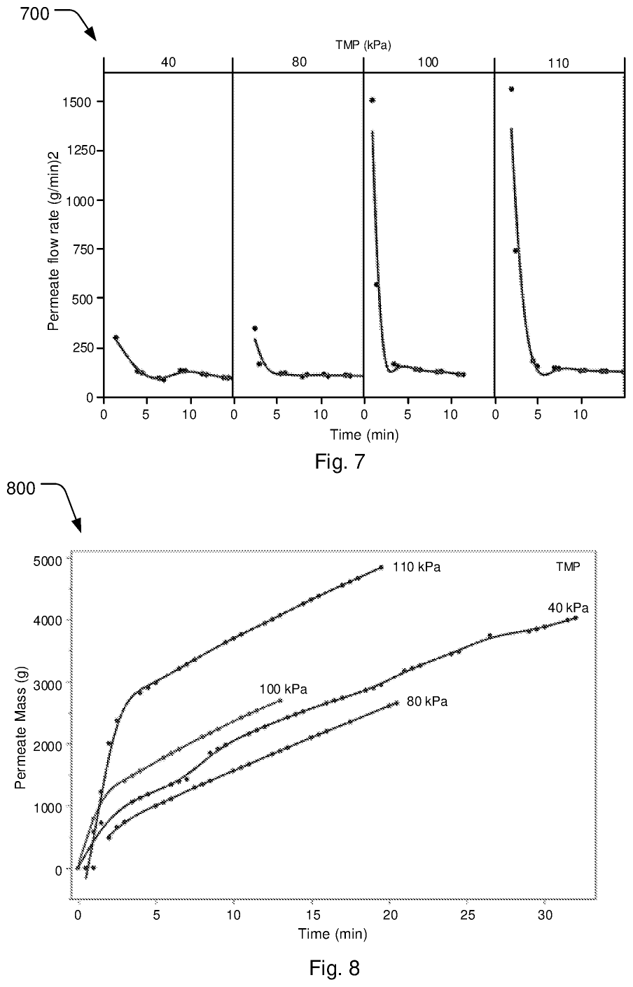

FIGS. 7-24 show results of experiments conducted with respect to various embodiments of the method 100 to remove the solids 41 from the solution 44.

### Example 1—Effects of Varying TMP

FIGS. 7 and 8 show the effects of varying TMP on permeate flow rate and permeate mass for a hemopexin extract (Fraction IV-4) with a 1:10 extraction ratio (3.64 kg Fraction IV-4:36.4 kg of 40 mM disodium phosphate) which was fed into a ceramic hollow fibre filter with a 0.6 micron pore size and a filter area of 0.2 m² and using a water bath with a copper cooling coil (not shown) to maintain the feed tank temperature below 25° C. Backwashing was also performed using 2 L of buffer per 2 L of permeate collected and the pump speed was set to 30 Hz (˜60-80 L/min).

FIG. 7 shows a graph 700 of experimental results of permeate flow rate versus time for different TMP values as recorded by a data logger. Under all trans-membrane pressures applied, the permeate had a higher initial flow rate, but rapidly achieved a steady state flow rate of 100-150 g/min (permeate flux ˜45-75 L/m²/h) after approximately 3 minutes.

FIG. 8 shows a graph 800 of experimental results of permeate mass versus time for different TMP values as recorded by the data logger. These results show that in general, higher TMP values resulted in higher initial permeate flux, though all conditions had reached similar steady permeate flux over time. The 40 kPa TMP had a higher initial flux than the 80 kPa TMP, which was possibly due to the 40 kPa condition being performed first and therefore the filter membrane had minimal fouling at that point.

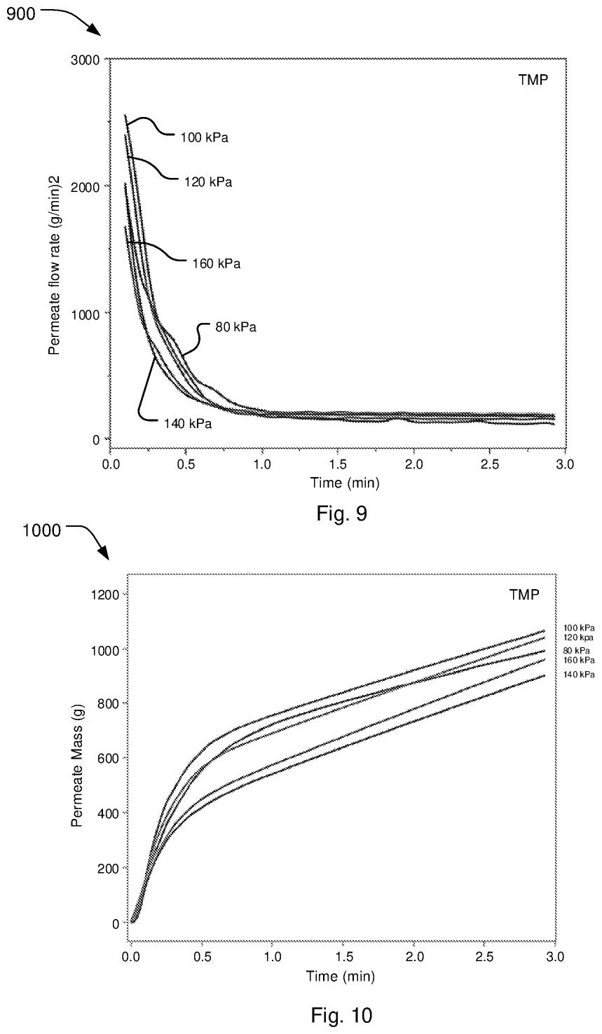

FIGS. 9 and 10 show the effects of varying TMP on permeate flow rate and permeate mass for a hemopexin extract (Fraction IV-4) with a 1:2.5 extraction ratio which was fed into a ceramic hollow fibre filter with a 0.6 micron pore size and a filter area of 0.2 m² and using the water bath to maintain the feed tank temperature at below 25° C. Backwashing was also performed using 2 L of buffer per 2 L of permeate collected.

FIG. 9 shows a graph 900 of experimental results of permeate flow rate versus time and FIG. 10 shows a graph 1000 of experimental results of permeate mass versus time for different TMP values as recorded by a data logger. Under all trans-membrane pressures applied, the permeate had a higher initial flux, but rapidly achieved a steady state flow rate of 150-200 g/min (permeate flux ˜45-75 L/m²/h) after approximately 0.8 minutes. The reduction of permeate flow rate was more rapid than the experiment using a 1:10 extraction ratio as described above, while the final stabilised permeate flow rate was similar. The graphs 900, 1000 suggest that there is no significant difference of the initial permeate flow rate among different TMP conditions. Also, it seems that higher TMP values (140 and 160 kPa) may yield lower initial flux, therefore, lower TMP values (≤120 kPa) may be used in the case of 1:2.5 extract.

The graphs 900, 1000 were generated based on an average of 4-5 runs of the experiment and it was found that the initial flux varied among the runs at the same TMP condition. Such variation could be due to the continuous manual adjustment of the permeate valve to control the TMP at the beginning of the experiment. It was also found that a cake/gel layer was likely to form during the initial transient-state of the filtration. The cake formation resulted in a reduction in permeate flow rate as well as a reduction in protein transmission, which was not able to be addressed by higher TMP values.

Table 1 below summarises the results from the above hemopexin experiments and provides a comparison of results between the 1:2.5 hemopexin extract and the 1:10 hemopexin extract. The results show a total hemopexin recovery of 71% obtained following the filtration of both hemopexin extracts, indicating that a recovery of over 70% is attainable using a lower extraction ratio for hemopexin.

TABLE 1
Mass, concentration and recovery results of the
hemopexin extract (feed) and final permeate.

| | | 1:10 | 1:2.5 |
|---|---|---|---|
| Type | Property | Extract | Extract |
| Feed | Mass (kg) | 40 | 29.5 |
| | Hemopexin concentration (total, g/L) | 0.875 | 1.988 |
| | Hemopexin concentration | 0.760 | 1.810 |
| | (uncomplexed, g/L) | | |
| Permeate | Mass (kg) | 64.94 | 49.94 |
| | Hemopexin concentration (total, g/L) | 0.384 | 0.838 |
| | Hemopexin concentration | 0.280 | 0.720 |
| | (uncomplexed, g/L) | | |
| Recovery | Total hemopexin recovery | 71% | 71% |
| | Uncomplexed hemopexin recovery | 60% | 67% |

### Example 2—Effects of Varying Pump Speed

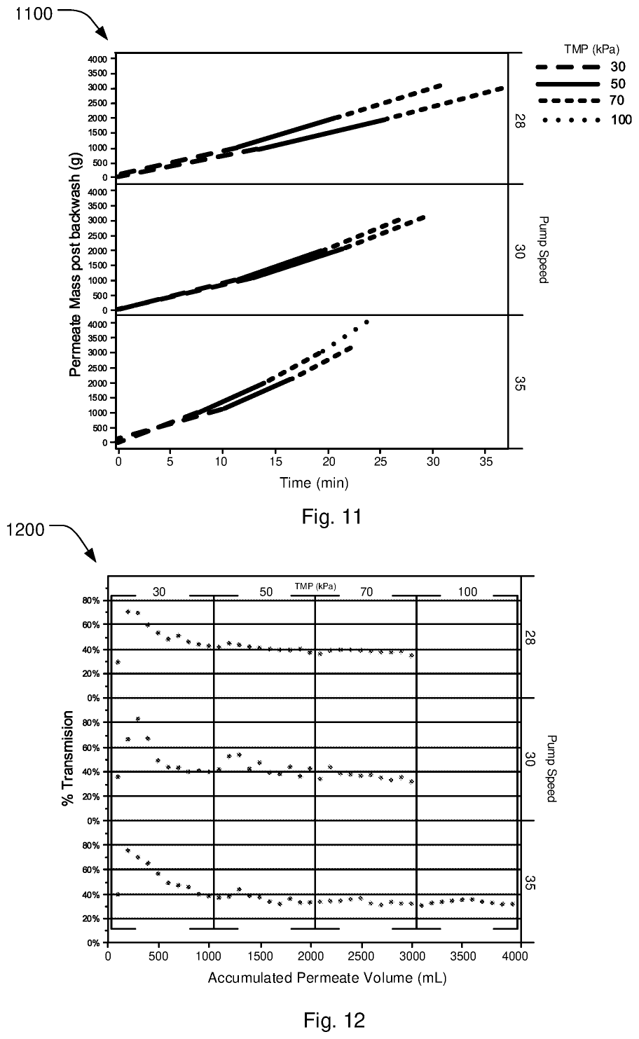

FIGS. 11 and 12 show the effects of varying pump speed for different TMP values. The experiments were performed using a hemopexin extract (Fraction IV-4) with a 1:10 extraction ratio (3.64 kg Fraction IV-4:36.4 kg of 40 mM disodium phosphate) which was fed into a ceramic hollow fibre filter with a 0.25 micron pore size and a filter area of 0.2 m² and using the water bath to maintain the feed tank temperature below 25° C.

FIG. 11 shows a graph 1100 of experimental results of permeate mass versus time for varying pump speeds across different TMP values. Runs were performed until 3 L of permeate was recovered and backwashes were performed between each run. Compared to the previous example, the initial high permeate flow rate was not present. Also, the higher the pump speed and/or the TMP, the higher the permeate flow rate.

FIG. 12 shows a graph 1200 of protein transmission percentage versus accumulated permeate volume for varying pump speeds across different TMP values. The protein transmission percentage was initially high (˜80%) but gradually reduced to a steady-state of ˜40% at 30 kPa TMP, regardless of the pump speed. The ramp-up of TMP did not lead to a further reduction in protein transmission, and the pump speed had no significant effect on the initial reduction rate of protein transmission. Therefore, the results suggest that low TMP and high pump speed cannot prevent cake formation on the membrane. However, it also indicates that the low protein transmission can be potentially resolved by performing backwash for every 0.5 L of permeate produced to maintain an overall protein transmission of greater than 50%.

### Example 3—Effects of Backwash Volume

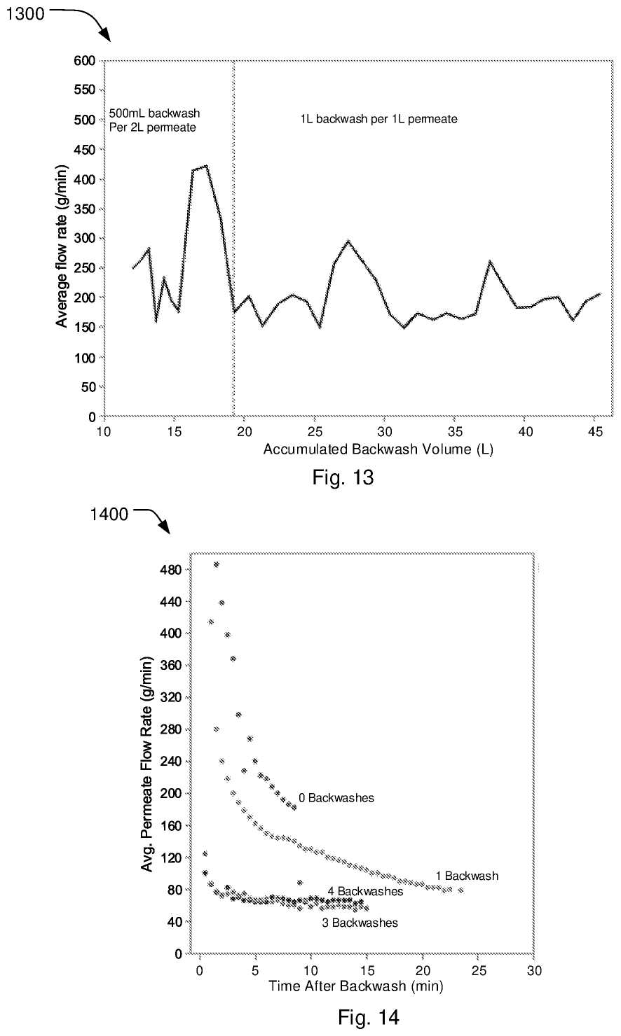

FIG. 13 shows the effects of backwash volume on average permeate flow rate and Table 1 shows the results of hemopexin concentration and recovery. In this experiment, the same hemopexin extract as Example 2 was used which was fed into a ceramic hollow fibre filter with a 0.6 micron pore size and a filter area of 0.2 m² and a trans-membrane pressure of 110 kPa. The same water bath was used as in the previous example.

FIG. 13 shows a graph 1300 of average permeate flow rate versus accumulated backwash volume. For approximately the first 19 L of accumulated backwash, backwashing was performed using 500 mL of buffer per 2 L of permeate collected in order to concentrate the solution to half the volume. Backwashing was then performed using 1 L of buffer per 1 L of permeate. The large variations in average permeate flow rate were likely due to variations in time spent backwashing and opening and closing valves in between backwashing cycles. However, the average flow rate was overall relatively stable at 150-250 g/min for both backwashing conditions. Hence, a backwash volume of 500 mL is demonstrated to be sufficient to clean the filter membrane under these conditions.

Hemopexin complex was measured during this experiment using SEC-HPLC and hemopexin concentration was measured using RP-HPLC. Table 1 shows the mass, total hemopexin concentration, hemopexin complex percentage and recovery in the hemopexin extraction prior to the feeding or the filtering, the final retentate and the final permeate.

### Example 4—Using Albumin and IgG Extracts and the Effects of Backwashing

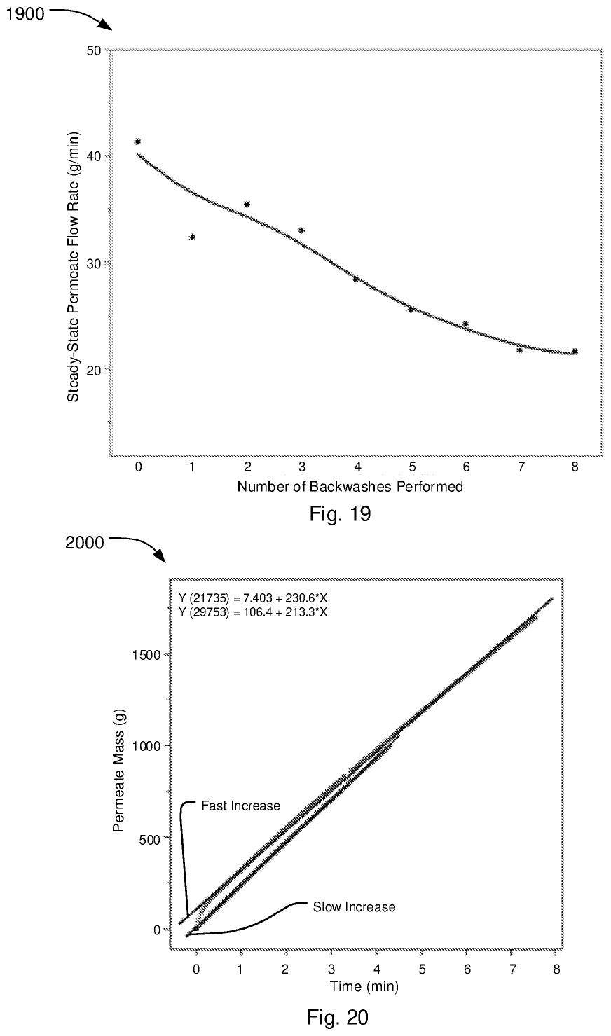

FIGS. 14-17 show the effects of backwashing on albumin extract (Fraction V) and FIGS. 18-19 show the effects of backwashing on IgG extract (Fraction I+II+III).

The experiments relating to the results shown in FIGS. 14-17 were conducted with an extraction ratio of 1:2 (6.7 kg Fraction V: 13.3 kg 10 mM sodium acetate) using a ceramic hollow fibre filter with a pore size of 0.25 microns and a filter area of 0.2 m². The pump speed was set to 35 Hz (˜90 L/min feed rate) and the TMP was set to 100 kPa. Backwashing was performed using 1 litre of buffer per litre of permeate produced. A water bath with a copper cooling coil attached was used to maintain the feed tank temperature below 25° C.

FIG. 14 shows a graph 1400 of average permeate flow rate (across 2 runs) versus time after backwash for different numbers of backwashes performed. The permeate flow rate had a clear decreasing trend over time at the early stage of filtration (i.e. before the third backwash). During this stage, the initial permeate flow was greater than 400 g/min and the flow rate gradually reduced over a time span of 20 minutes to approximately 80 g/min at which the next backwash was performed. However, at the later stage of filtration, the initial high permeate flow rate became less apparent and the permeate flow almost immediately reached a steady-state flow rate of approximately 60 g/min.

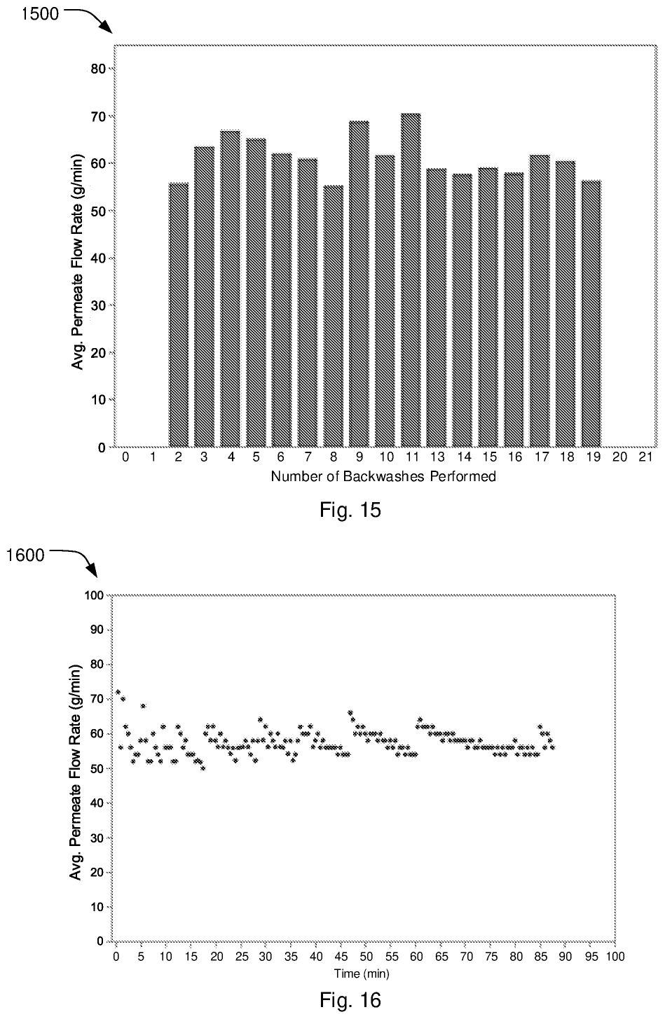

FIG. 15 shows a graph 1500 of average permeate flow rate (across 2 runs) versus number of backwashes performed to investigate the changing flow rate between backwash cycles. The flow rate results of the early filtration runs (with 0 and 1 backwash performed) were omitted, since their behaviours differed significantly from the others. The average permeate flow rate was consistent and stable at 55-65 g/min over the course of the experiment. This equates to a permeate flux of 18 L/m²/h. The flow rate was significantly less than the average flow rate observed (approximately 200 g/min) in Example 2 under the same operating conditions.

FIG. 16 shows a graph 1600 of average permeate flow rate versus time for an extended filtration without backwash over 90 min, in order to investigate the trend of permeate flow rate over a longer period of time. The results show that the permeate flow remained stable at 50-60 g/min, which is consistent with the average permeate flow rate observed in FIGS. 14 and 15.

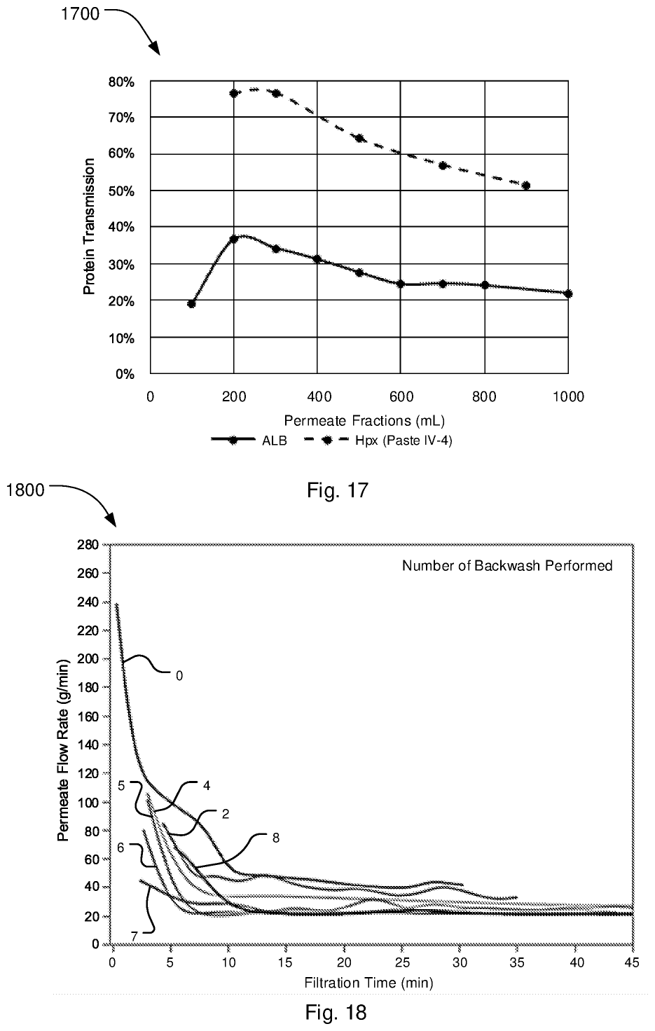

FIG. 17 shows a graph 1700 of protein transmission versus permeate fractions in which protein transmission was measured at particular permeate fractions for the albumin extract compared to the hemopexin extract from Example 2. It is observed that the protein transmission in the case of hemopexin extract was overall higher than in the albumin extract.

Table 2 shows the mass, total albumin concentration and recovery in the albumin extraction prior to the feeding or the filtering, the final retentate and the final permeate. Albumin concentration was measured using nephelometry. The final permeate turbidity was 4.63 NTU.

TABLE 2
Mass, concentration and recovery results of the albumin
extract, final retentate and final permeate

| | Albumin extract | Final retentate | Final permeate |
|---|---|---|---|
| Mass (kg) | 20 | 22.6 | 32 |
| Albumin | 128 | 55.2 | 40.8 |
| concentration (g/L) | | | |
| Recovery | N/A | 49% | 51% |

The experiments relating to the results shown in FIGS. 18-19 were conducted with an extraction ratio of 1:4.5 (3.6 kg Fraction I+II+III: 16.4 kg 0.22M sodium acetate) using a ceramic hollow fibre filter with a pore size of 0.25 microns and a filter area of 0.2 m². The pump speed was set to 32 Hz (˜80 L/min feed rate) and the TMP was set to 80 kPa. Backwashing was performed using 1 litre of buffer per litre of permeate produced. A water bath with a copper cooling coil attached was used to maintain the feed tank temperature below 25° C.

FIG. 18 shows a graph 1800 of permeate flow rate versus time after backwash for different numbers of backwashes performed. The permeate flux decayed similarly to the albumin extract results. At the early stage of the filtration, the initial permeate flow rate was higher (i.e. greater than 200 g/min) and the permeate flow rate gradually reduced over time. At the later stage of filtration (i.e. after the fifth backwash), the initial high permeate flow rate became less apparent and the permeate flow almost immediately reached a steady-state flow rate.

FIG. 19 shows a graph 1900 of steady state permeate flow rate versus number of backwashes performed. Different from the previous filtration experiments of hemopexin extract and albumin extract, the steady state flow rate reduced over the course of the experiment. It suggested that the membrane of the hollow fibre filter was blocked by foulant and the blockage could not be removed by backwashes. During the experiment, a backpressure of 5 bar was applied on the permeate side during the backwashes, which was higher than the typical backpressure of 4 bar in the other experiments. However, it still failed to provide a more effective backwash. It was likely that the foulant was strongly adsorbed to the membrane.

### Example 5—Effects of Varying TMP Ramp-Up Rate

FIG. 20 shows a graph 2000 of permeate mass versus time for a fast increase or ramp-up of TMP and a slow increase or ramp-up of TMP to 100 kPa using the ceramic hollow fibre filter with a pore size of 0.25 microns and a filter area of 0.2 m². The pump speed was set to 35 Hz (˜90 L/min feed rate) and a water bath with a copper cooling coil attached was used to maintain the feed tank temperature below 25° C. The fast increase reached 100 kPa in under 10 seconds and the slow increase reached 100 kPa in approximately 30 seconds. There was no significant difference in the permeate flow rates between the slow and fast ramp-up.

### Example 6—Effects of Varying Solution pH

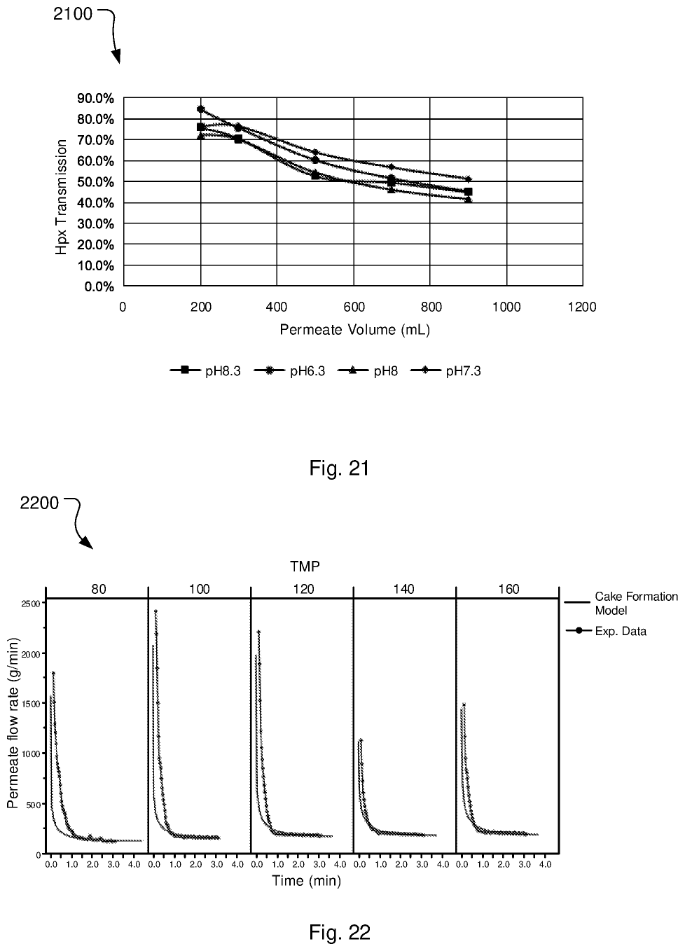

FIG. 21 shows a graph 2100 of hemopexin transmission versus permeate volume for different pH levels of the solution. The experiment was performed using a hemopexin extract (Fraction IV-4) with a 1:10 extraction ratio (3.64 kg Fraction IV-4:36. 4 kg of 40 mM disodium phosphate) which was fed into a ceramic hollow fibre filter with a 0.25 micron pore size and a filter area of 0.2 m² and using the water bath to maintain the feed tank temperature at below 25° C. The trans-membrane pressure was set to 30 kPa and the pump speed was set to 35 Hz (˜90 L/min feed rate). The graph 2100 indicates minimal effect of pH on hemopexin transmission across the membrane.

### Example 7—Fouling Model Investigation

FIGS. 22-24 show results of the investigation of how the experimental data produced from hemopexin, IgG and albumin extracts compares with the theoretical results from the standard/internal blocking model, the intermediate/partial blocking model and the cake filtration/formation model.

FIG. 22 shows a graph 2200 of permeate flow rate versus time to compare smoothed experimental permeate flow rate curves with the theoretical curve generated from the cake fouling model which most closely fitted the experimental results. Hemopexin extract was used under the same conditions as Example 1 across the different TMP values and the similarity between the experimental and theoretical curves indicates that for this experiment, the rapid reduction of permeate flow rate was likely due to cake formation.

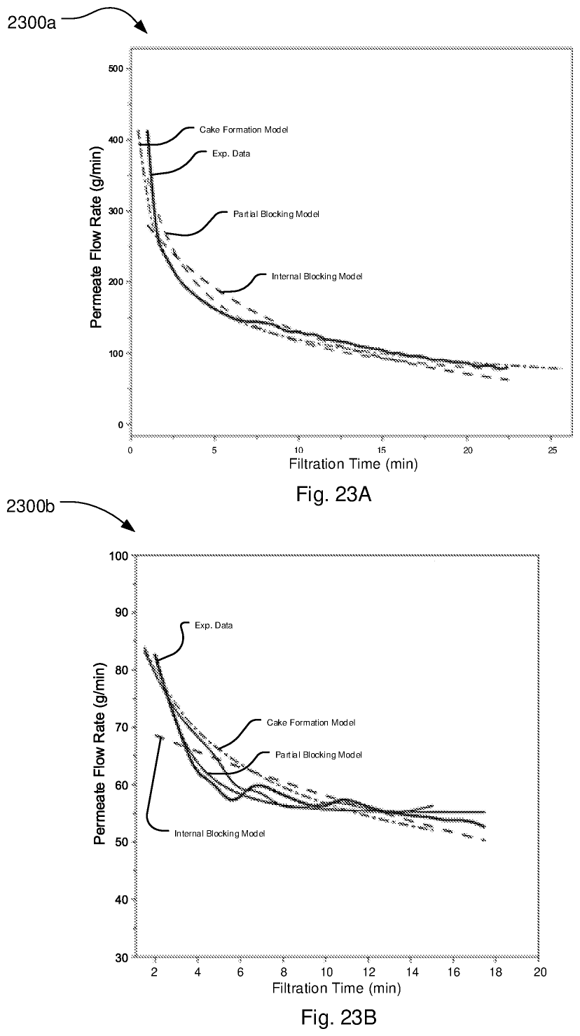

FIGS. 23A and 23B show graphs 2300a and 2300b, respectively, of permeate flow rate versus time to compare smoothed permeate flow rate curves from using albumin extract as in Example 4 with the theoretical curves generated from the cake, partial and internal fouling models. FIG. 23A shows experimental results from earlier in the experiment of Example 4 (after the first backwash) and FIG. 23B shows experimental results from later in the experiment of Example 4 (after the thirteenth backwash). It appears that both in the early and late stages of the experiment, the same fouling mechanism, being partial fouling, was causing the permeate flow curve behaviour. The partial blocking mechanism suggests that the foulant is attached to both the membrane surface and the membrane pore (as shown in FIG. 4C). The blocking and unblocking of the pore can occur dynamically, and the backwashing between the filtration was likely to remove only a small portion of the foulant attached on the membrane. As a result, the steady-state permeate flow gradually reduced at the early stage of the filtration and became relatively stable at the later stage of filtration.

Given that the protein transmission rate was only 20-40% in this experiment and the albumin concentration in the feed tank was high as shown in Table 2, it was likely that a gel layer was also formed on top of the filter membrane. However, this gel layer did not have a dominant effect in the decay of permeate flow. It was demonstrated in the literature that at lower pH levels of the solution (3-5), there was greater adsorption of albumin on silicon carbide materials, and ceramic membrane was more rapidly fouled by albumin, compared with neutral pH as used in this experiment.

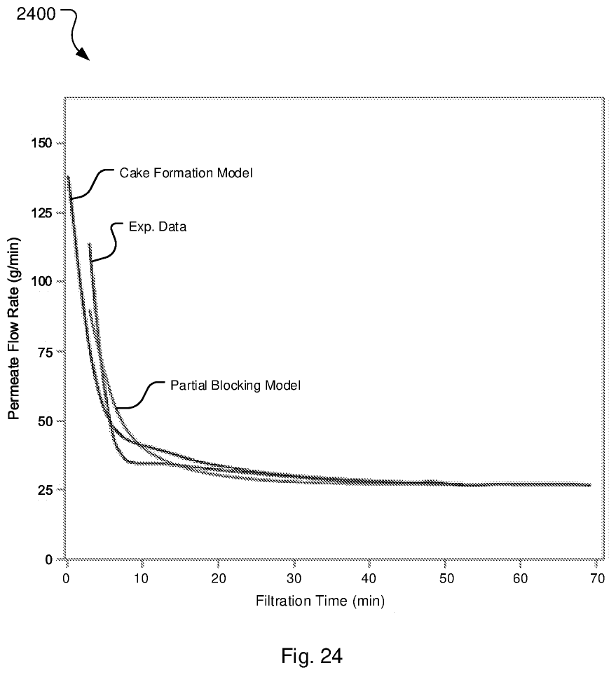

FIG. 24 shows a graph 2400 of permeate flow rate versus time to compare smoothed experimental permeate flow rate curves from using IgG extract as in Example 4 with the theoretical curve generated from the cake fouling and partial blocking models which most closely fitted the experimental results. It is possible that both pore blocking mechanisms were involved in the permeate flux decay, but the reduction in steady-state permeate flux was likely due to the partial pore blocking. Partial blocking occurs when the foulant has a size greater than the pore size, such that it could not enter the pore and become attached to the membrane surface, which partially blocking the membrane pore. In the filtration of IgG extract, the foulant was likely to be the lipid/lipoprotein aggregates of which the average size is greater than the membrane pore size of 0.25 microns as used in the experiment.

1. A method of filtering solids from a solution derived from blood plasma, the method comprising:
feeding the solution into a hollow fibre filter at a feed rate, the hollow fibre filter comprising a plurality of hollow fibres, each hollow fibre comprising a membrane defining an elongate hollow fibre channel; and
filtering the solution using the hollow fibre filter to produce a permeate and a retentate, the permeate passing through pores of the membrane at a trans-membrane pressure and the retentate flowing from respective outlets of the elongate hollow fibre channels, wherein the permeate has a reduced solids content with respect to the solution fed into the hollow fibre filter.

2. The method of claim 1, further comprising recycling the retentate into the solution for feeding into the hollow fibre filter.

3. The method of claim 1 or claim 2, wherein the feed rate is defined by a cross flow velocity of from about 0.6 m/s to about 4.0 m/s.

4. The method of any one of the preceding claims, wherein the pores of the membrane have an average pore size of from about 0.1 microns to about 4 micron.

5. The method of any one of the preceding claims, wherein the trans-membrane pressure is from about 20 kilopascals to about 300 kilopascals.

6. The method of any one of the preceding claims, further comprising measuring a permeate flux during filtering.

7. The method of claim 6, wherein the permeate flux is at least about 3 litres per square metre of the hollow fibre filter area per hour.

8. The method of any one of the preceding claims, wherein the method comprises multiple filtering steps, and wherein the method further comprises feeding a backwash solution through the hollow fibre filter between filtering steps.

9. The method of claim 8, wherein the backwash solution comprises a buffer solution.

10. The method of claim 8 or claim 9, wherein the backwash solution comprises permeate.

11. The method of any one of claims 8 to 10, wherein the volume of backwash solution fed to the hollow fibre filter between filtering steps is provided in a ratio to the permeate volume obtained during the filtering step prior to backwash of from about 1:6 to about 1:1.

12. The method of any one of the preceding claims, wherein the solution has a conductivity of about 2 mS/cm to about 40 mS/cm.

13. The method of any one of the preceding claims, wherein the solution has a conductivity of about 8 mS/cm to about 15 mS/cm.

14. The method of any one of the preceding claims, wherein the solution is at a temperature of from about 4° C. to about 37° C.

15. The method of any one of the preceding claims, wherein the solution comprises a blood plasma fraction.

16. The method of any one of the preceding claims, wherein the solution comprises a buffer.

17. The method of claim 16, wherein the buffer comprises sodium acetate or a phosphate.

18. The method of claim 16 or claim 17, wherein the solution has an extraction ratio of kilograms of blood plasma fraction to kilograms of buffer of from about 1:2 to about 1:20.

19. The method of any one of the preceding claims, wherein the solution comprises hemopexin.

20. The method of any one of claims 1-19, wherein the solution comprises albumin.

21. The method of any one of claims 1-19, wherein the solution comprises immunoglobulin G.

22. The method of claim 21, further comprising adding octanoic acid to the permeate for delipidating the permeate.

23. The method of any one of the preceding claims, wherein the solution comprises a filter aid.

24. The method of any one of the preceding claims, wherein the solution has a pH of between about 4 and about 9.

25. The method of any one of the preceding claims, wherein a recovery of the permeate is at least 30%.

26. The method of any one of the preceding claims, wherein a recovery of the permeate is at least 50%.

27. The method of any one of the preceding claims, wherein a recovery of the permeate is at least 75%.

28. The method of any one of the preceding claims, wherein a recovery of the permeate is at least 90%.

29. The method of any one of the preceding claims, wherein a turbidity of the permeate is less than about 400 nephelometric turbidity units.

30. A blood plasma product produced using the method of any one of the preceding claims.
